(19) United States
(12) Patent Application Publication
(10) Pub. No.: US 2015/0122895 A1
(43) Pub. Date: May 7, 2015

(54) TRANSPONDER PATCHES

(71) Applicant: Prosec Protection Systems, Inc., Freehold, NJ (US)

(72) Inventor: Mark Kosloski, Jackson, NJ (US)

(21) Appl. No.: 14/536,096

(22) Filed: Nov. 7, 2014

Related U.S. Application Data
(60) Provisional application No. 61/901,144, filed on Nov. 7, 2013.

(51) Int. Cl. G06K 19/077; A61B 5/00; A61B 5/117

(52) U.S. Cl. CPC G06K 19/0776; A61B 5/117; A61B 5/6833
USPC 235/492; 428/34.1

(57) ABSTRACT

Transponder patch devices are disclosed which include at least one protective layer, a host-contacting layer having an adhesive disposed on a first surface of the at least one protective layer, and at least one padding layer disposed between the at least one protective layer and the host-contacting layer, wherein the transponder is positioned within the padding layer. Patches suitable for containing transponders are also disclosed.

### CROSS-REFERENCE TO RELATED APPLICATION

This application claims the benefit of U.S. Provisional Application No. 61/901,144 filed Nov. 7, 2013, the entirety of which is incorporated herein by reference.

### FIELD OF THE INVENTION

The invention relates to transponders. More specifically, it relates to transponder adhesive patches.

### BACKGROUND OF THE INVENTION

RFID devices use electromagnetic fields to transfer data for the purpose of identifying and tracking transponders containing electronically stored information. The transponders may be powered by electromagnetic induction from magnetic fields produced near a reader. Passive type transponders collect energy from interrogating radio waves of a reader/controller. Other types such as active type transponders may include a power source such as a battery and may transmit independent of a reader/controller and may operate at hundreds of meters from the reader.

### SUMMARY OF THE INVENTION

The present invention relates to a patch configured to include a transponder device. Devices in accordance with the present disclosure permit economic reuse of transponders. Currently, transponders used in the healthcare industry require secure hardware carriers. Devices in accordance with the present disclosure provide disposable adhesive patches which may include integral transponders. In other embodiments patches are provided which permit insertion and removal of transponders wherein the transponders are securely fixed to a host via the patch.

In one embodiment a device includes a patch such as but not limited to an adhesive patch and a transponder. The device may further include one or more sensors positioned to contact a surface of a host.

In one embodiment a patch includes a host-contacting layer including an adhesive to adhere the device to the host, one or more a protective layers, one or more padding layers and a cavity or pocket for containing a transponder; and optionally, a release liner for protecting the adhesive layer prior to deployment. Removal of the release liner exposes the adhesive layer so that the device may be applied to the host. In another embodiment the device includes the transponder.

In another embodiment a patch includes a conduit in communication with the cavity to permit insertion of a transponder into the cavity. The conduit may be formed through the protective layer and the padding. A flap, securable with an adhesive, may be disposed over the open end of the conduit to prevent the entry of contaminants such as moisture, dirt, etc. into the cavity.

In another embodiment a conduit is formed between the cavity and a surface of a release liner, so that removal of the release liner permits insertion of a transponder into the cavity via the conduit, and fixation of the patch to the host effectively closes the opening of the conduit. Upon removal of the patch, the transponder may be removed from the cavity via the conduit, which is open by virtue of the removal of the patch from the host.

Devices as disclosed herein may be fixed to any host and used in conjunction with readers/controllers known in the art.

### BRIEF DESCRIPTION OF THE DRAWINGS

So that those having ordinary skill in the art will have a better understanding of how to make and use the disclosed systems and methods, reference is made to the accompanying figure wherein:

FIG. 1 is top plan view of a device according to an embodiment of the present disclosure;

FIG. 1A is a schematic cross-sectional view of a device taken along the line A-A′ of FIG. 1 according to an embodiment of the present disclosure;

FIG. 1B is a schematic cross-sectional view of a device taken along the line A-A′ of FIG. 1 according to another embodiment of the present disclosure;

FIG. 2 is a schematic cross-sectional view of a device according to an embodiment of the present disclosure;

FIG. 3 is a schematic cross-sectional view of a device according to an embodiment of the present disclosure;

FIG. 3A is a perspective view of a transponder insertable in a conduit of the device according to FIG. 3;

FIG. 3B is a perspective view of a transponder with leads and a sensor insertable in a conduit of the device according to FIG. 3;

FIG. 4 is a schematic view of a device in accordance with an embodiment of the present disclosure affixed to pediatric or adult host;

FIG. 5 is a schematic view of a device in accordance with an embodiment of the present disclosure affixed to an infant host;

FIG. 6 is a schematic view of a device in accordance with an embodiment of the present disclosure affixed to an inanimate host; and

FIG. 7 is a schematic view of a device affixed to a host in proximity of a reader/controller in accordance with an embodiment of the present disclosure.

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

It will be understood that when an element is referred to as being “coupled” or “connected” to another element, it can be directly coupled or connected to the other element or intervening elements may also be present. In contrast, when an element is referred to as being “directly coupled” or “directly connected” to another element, there are no intervening elements present. Like numbers refer to like elements throughout. As used herein the term “and/or” includes any and all combinations of one or more of the associated listed items.

In addition, spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Well-known functions or constructions may not be described in detail for brevity and/or clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein. Unless otherwise indicated or defined, all technical and scientific terms used herein have the same meaning as commonly understood by one of ordinary skill in the art to which the invention pertains. The terminology used herein is for describing particular embodiments only and is not intended to be limiting.

Transponders disclosed herein may be any transponder known in the art operable to monitor the presence of a host Exemplary transponders include but are not limited to transponders available from Stanley Healthcare of Waltham, Mass.; McRoberts Security Technologies of Freehold, N.J.; Centrak, Inc. of Newtown, Pa.; and Sonitor US of Stamford, Conn.. As used herein the term “host” may refer to any person, such as a patient, a staff member, visitor, or the like; or an article, item, asset or the like, to which the transponder may be affixed or attached.

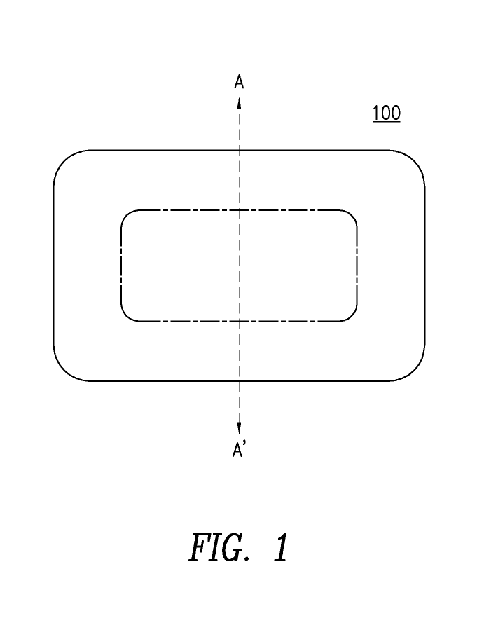

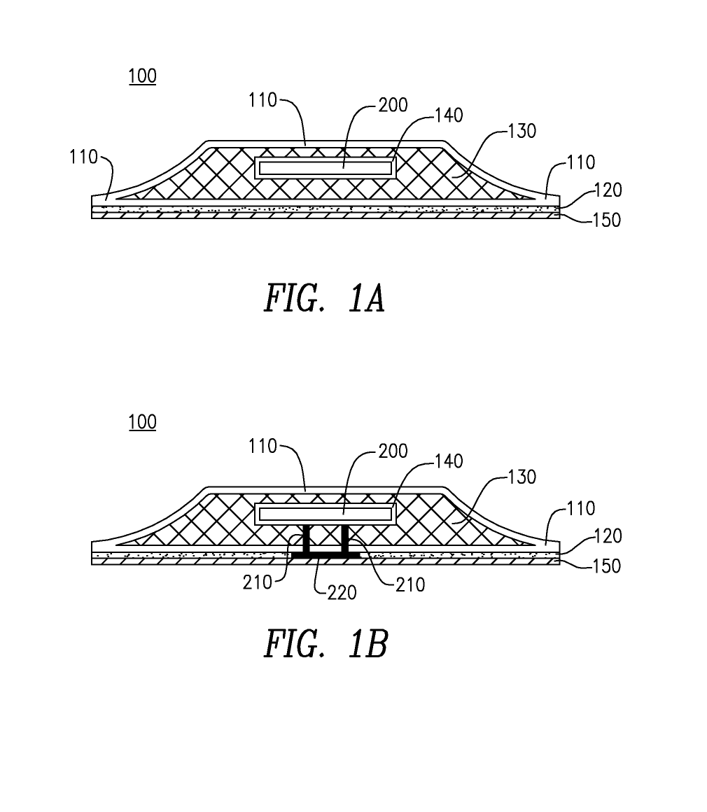

Now referring to FIG. 1, a device 100 is shown. With further reference to FIG. 1A a device 100 includes a protective layer 110, an adhesive layer 120 disposed on a protective layer 110, a padding layer 130, a cavity 140 for containing a transponder 200, an optional release liner 150 for protecting the adhesive layer 120 prior to deployment, and a transponder 200 disposed in the cavity 140. Removal of the release liner 150 exposes the adhesive layer 120 so that the device may be applied to the host.

The adhesive layer 120 constitutes a host-contacting layer. The adhesive layer may include any suitable adhesive well-known in the art, such as those adhesives used for bandages, wound dressings and the like.

The protective layer 110 may be any suitable material operable to protect the transponder 200 from damage. For example, the protective layer 110 may be but is not limited to, fabric, plastic, polyester, polyurethane, polyvinyl, PEEK, fiberglass, Kapton, foil, reinforced paper, coated paper or the like, or any combination thereof. The protective layer 110 may have any thickness that is suitable for the particular application for which the presently disclosed subject matter is employed. In one or more embodiments the backing substrate has a thickness of about 0.01 to about 1.0 mm. The protective layer 110 may be permeable or semipermeable to liquid and/or gas, or may be liquid and/or gas impermeable. Additional protective layers 110 may be included in the device 100. One or more intermediate layers of protective layer 110 may be employed. In addition, while FIG. 1 (and the remaining embodiments) depict a single protective layer 110 which surrounds the padding layer 130, the protective layer may be formed of separate layers. For example, the portion of the protective layer 110 upon which adhesive layer 120 is disposed may be a separate protective layer 110.

Those skilled in the art will appreciate from the disclosure herein that the details presented will readily enable a skilled artisan to employ one or more methodologies for applying layer 104 by applying well-known layering techniques.

The padding layer 130 may be adhered or otherwise laminated to the protective layer 110 as shown. The padding layer 130 may be but is not limited to a synthetic or natural material, nylon, polyester, cellulose, sponge, knit or woven fibers, brushed or blended fibers, etc. or combinations thereof. The padding layer 130 provides damage protection, resistance from water and/or humidity, and provides a barrier to the skin of a host or surface of an article.

The cavity 140 may be disposed within the padding layer 130. The cavity 140 may be a hollow shell formed of acrylic, polyethylene, PVC or the like. Alternatively, a protective layer of the materials referenced above with respective to protective layer 110 may form one or more walls of cavity 140 to provide a boundary between the padding layer 130 and the cavity 140.

Further layers may be disposed in the device 100 on the interior of the cavity 140 or on the exterior of the cavity 140 to provide additional support or structure for protecting the transponder 200.

In another embodiment the transponder 200 may be disposed between plural layers of material of the device 100, retained in a pocket formed in or on the patch, encapsulated on or in a surface of the patch or the like.

Now referring to FIG. 1B, a device 100 may further include one or more sensors 220 operably connected to a transponder 200. The sensor 220 is positioned on a host-facing side of the device 100. In one embodiment the device 100 includes a release liner 150, which upon removal, exposes the sensor 220 so that upon application of the device 100 to the host, the sensor 220 contacts the surface of the host. The sensors 220 may be but are not limited to any combination of skin, optical and/or mechanical sensors known in the art to ensure the device 100 and transponder 200 are constantly affixed securely to the host. In the event the tag is either tampered with or removed, the sensor(s) 220 may emit a tamper beacon to a monitoring system and generate an alert/alarm for a custodial entity to respond accordingly.

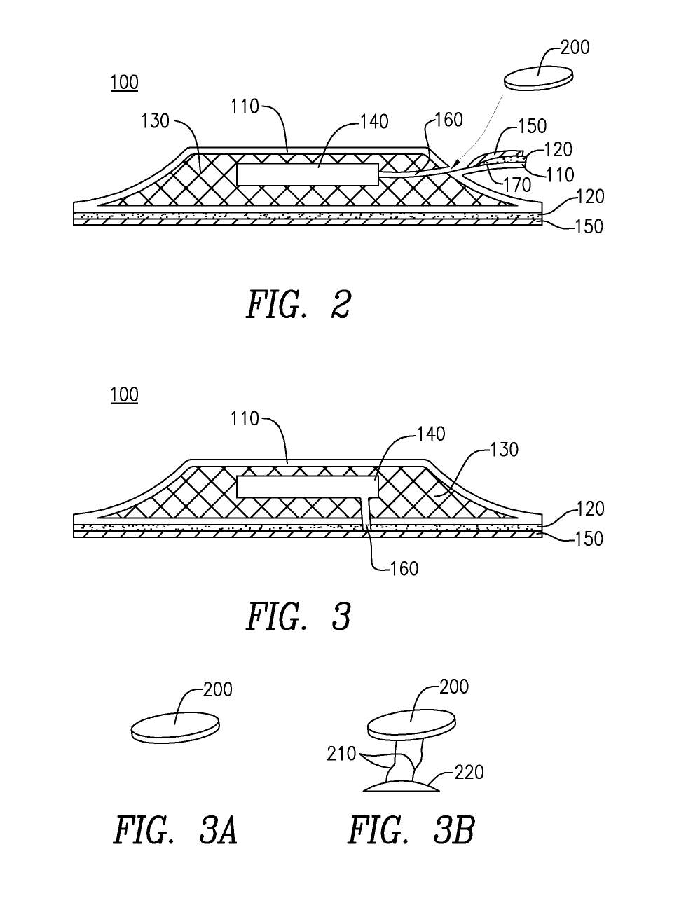

Now referring to FIG. 2, in another embodiment a device 100 includes a conduit 160 in communication with the cavity 140 to permit insertion of a transponder 200 into the cavity 140 as shown. The conduit may be formed through the protective layer 110 and the padding layer 130. Flap 170, having an adhesive 120 and release liner 150, extending from the surface of the protective layer 110, may be deployed to close the opening of the conduit at the protective layer 110/air interface to prevent the entry of contaminants such as moisture, dirt, etc. into the cavity 140. In another embodiment the device 100 does not include flap 170.

Now referring to FIG. 3, in another embodiment a conduit 160 is formed between the cavity 140 and a surface of a release liner 150, so that removal of the release liner 150 exposes an opening of the cavity to permit insertion of a transponder 200 as shown in FIG. 3A into the cavity 140 via the conduit 160. Fixation of the device 100 to the host effectively closes the opening of the conduit 160. Upon removal of the device 100, the transponder 200 may be removed from the cavity 140 via the conduit 160, which is open to the air by virtue of the removal of the device 100 from the host. With further reference to FIG. 3B, a transponder 200 including one or more sensors 220 may be inserted into cavity 140, with the leads 210 extending through the conduit 160, and a surface or surfaces of the sensor(s) 220 adhering to the adhesive layer 120 and positioned to contact a surface of a host.

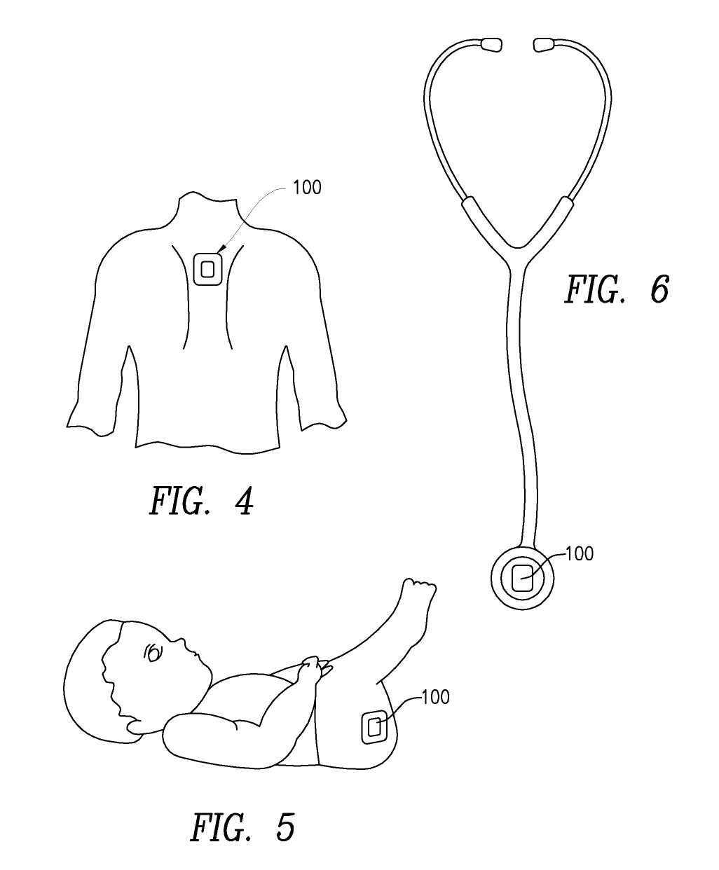

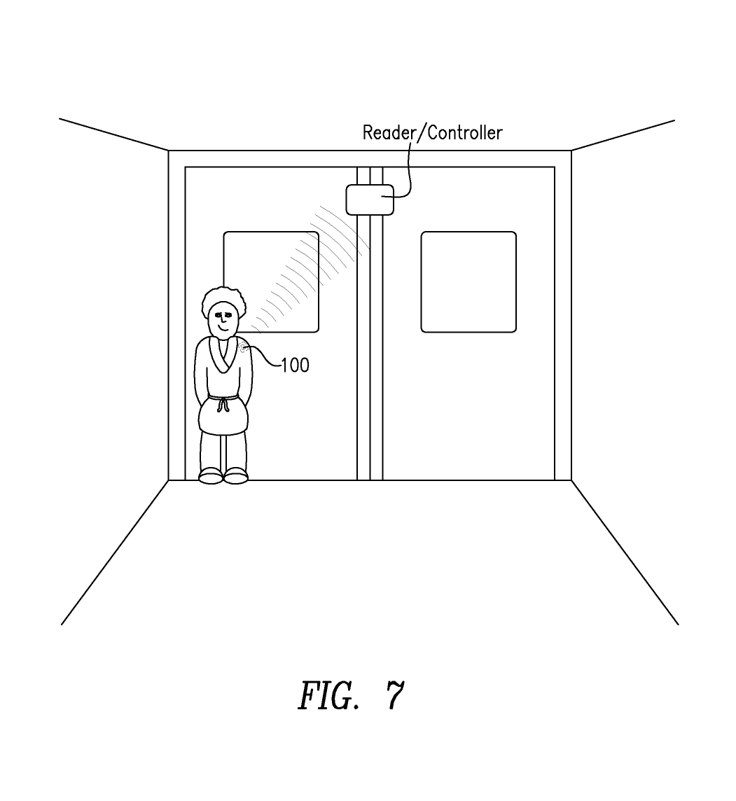

Now referring to FIGS. 4-6, the device 100 may be affixed directly to the skin of a patient or surface area of an object. With further reference to FIG. 7, devices as disclosed herein may be employed to secure the host to which it is attached by communicating with any suitable reader/controller device that may be programmed to restrict or grant access at an entry/egress point (referred to herein as a portal). Reader/controller devices are will known in the art. The installed reader/controller may interface with an access control system or device, or directly with a lock at the portal. The transponder 200 may communicate wirelessly with any associated receivers, readers, controllers, etc. via any single spectrum or combination of spectra of light, including but not limited to such as radio frequencies (RF), including but not limited to Wi-Fi, Zigbee, or other standard or proprietary RF; microwave, infrared, near infrared, visible, ultraviolet, X-ray, gamma ray, or high energy gamma ray spectra, and/or via mechanical wave such as but not limited to sound at any frequency or oscillation rate.

Although the apparatus and methods of the present disclosure have been described with reference to exemplary embodiments thereof, the present disclosure is not limited thereby. Indeed, the exemplary embodiments are implementations of the disclosed systems and methods are provided for illustrative and non-limitative purposes. Changes, modifications, enhancements and/or refinements to the disclosed systems and methods may be made without departing from the spirit or scope of the present disclosure. Accordingly, such changes, modifications, enhancements and/or refinements are encompassed within the scope of the present invention.

What is claimed is:

1. A device comprising an adhesive patch and a transponder.

2. The device according to claim 1 comprising at least one protective layer, a host-contacting layer comprising an adhesive disposed on a first surface of the at least one protective layer, and at least one padding layer disposed between the at least one protective layer and the host-contacting layer, wherein the transponder is positioned within the padding layer.

3. The device according to claim 2 further comprising a release liner releasably disposed on the host-contacting layer.

4. The device according to claim 1 further comprising at least one sensor operably connected to the transponder, wherein the at least one sensor is positioned to contact a surface of a host.

5. An adhesive patch comprising at least one protective layer, a host-contacting layer comprising an adhesive disposed on a first surface of the at least one protective layer, and at least one padding layer disposed between the at least one protective layer and the host-contacting layer, and a cavity positioned within the padding layer.

6. The adhesive patch according to claim 5 further comprising a conduit extending from the cavity to an outside surface of the device, the conduit operable to permit insertion of a transponder into the cavity through the conduit.

7. The adhesive patch according to claim 6 wherein the conduit is formed through the at least one padding layer and the at least one protective layer.

8. The adhesive patch according to claim 7 further comprising a flap extending from an outside surface of the patch positioned and operable to cover an opening formed at an end of the conduit at the surface of the patch.

9. The adhesive patch according to claim 8 wherein the flap comprises an adhesive layer formed on one side and a protective layer on an opposite side.

10. The adhesive patch according to claim 9 wherein the flap further comprises a release liner disposed on the adhesive layer,

11. The adhesive patch according to claim 5, further comprising a conduit extending from the cavity through the host-contacting layer, the conduit operable to permit insertion of a transponder into the cavity through the conduit.

12. The adhesive patch according to claim 11, the patch further comprising a release liner disposed on the adhesive layer.
